(19) United States
(12) Patent Application Publication
(10) Pub. No.: US 2025/0272429 A1
(43) Pub. Date: Aug. 28, 2025

(54) PLATFORM FOR MANAGING AUTHORIZATION REQUESTS

(71) Applicant: Basys.ai Inc., Boston, MA (US)

(72) Inventors: Dan Roth, Philadelphia, PA (US); Amber Nigam, Boston, MA (US); Jie Sun, Cambridge, MA (US)

(73) Assignee: Basys.ai Inc., Boston, MA (US)

(21) Appl. No.: 18/634,597

(22) Filed: Apr. 12, 2024

Related U.S. Application Data
(60) Provisional application No. 63/557,674, filed on Feb. 26, 2024.

(51) Int. Cl. G06F 21/62; G06F 21/31

(52) U.S. Cl. CPC G06F 21/6245; G06F 21/31

(57) ABSTRACT

An exemplary method for submitting an authorization request is provided. The method includes: authenticating a first user on the platform; creating or selecting a service request for a second user; retrieving requirements for the service request according to at least one policy of a second user; retrieving data that is pertinent to evaluating the at least one policy; predicting, using a machine learning model, the likelihood of the service request being denied or approved, wherein the machine learning model utilizes at least one guardrail to ensure the prediction of the likelihood of the service is accurate; determining whether the at least one policy and the data needs to be updated; updating the at least one policy and the data; reviewing the at least one policy and the data to determine all information is provided; and sending the service request to the second user.

### RELATED APPLICATION(S)

This application claims the benefit of U.S. Provisional Application No. 63/557,674, filed on Feb. 26, 2024. The entire teachings of the above application(s) are incorporated herein by reference.

### BACKGROUND

Prior authorization (PA) is a process that requires healthcare providers to obtain approval from insurance companies before prescribing certain medications, procedures, or services. The PA process is intended to ensure that the prescribed treatment is medically necessary and covered by the patient's insurance plan. However, the current PA process imposes a substantial administrative burden on providers due to the extensive volume of medical documentation and insurance policy text requiring submission. This often leads to care delays for patients and inefficiencies in the healthcare system.

In recent years, the issue of PA has garnered significant attention from key stakeholders in the U.S. healthcare industry. The U.S. Surgeon General, Centers for Medicare & Medicaid Services (CMS), Congress, 30 state legislatures, and the American Medical Association (AMA) have all identified PA as a primary source of administrative inefficiency. Despite this recognition, existing solutions have been unable to effectively streamline the PA process and alleviate the associated administrative burdens.

Traditional PA processing systems rely on static data models that cannot predictably determine certain characteristics of medical orders over time. This is because the conditions of medical orders change frequently, altering the relationships between elements of medical orders and related characteristics. As a result, healthcare providers are often faced with a fundamental dilemma: either accept an unacceptable delay in the processing of the medical order or risk improper medical order processing.

The application of generative AI to solve the PA problem is a novel approach. Basys.ai has developed a unique solution by training its own AI models to encode payer policies, defining guardrails and procedures to reduce errors, and creating custom word representations (so called embeddings) for its natural language processing (NLP) models that are specifically tailored to medical terminology and concepts. These models, trained on a dataset of 10 million patients of Mayo Clinic, operate with high accuracy in specialty disease areas, enabling the system to efficiently process PA requests and reduce administrative burdens for healthcare providers.

By leveraging advanced AI technologies and domain-specific training data, the basys.ai platform aims to revolutionize the PA process, ultimately improving patient care and reducing inefficiencies in the healthcare system.

### SUMMARY

According to one aspect of the subject matter described in this disclosure, an exemplary method for submitting an authorization request is provided. The method includes: authenticating a first user on the platform; creating or selecting, by the first user, a service request for a second user; retrieving requirements for the service request according to at least one policy of a second user; retrieving data that is pertinent to evaluating the at least one policy; predicting, using a machine learning model, the likelihood of the service request being denied or approved, wherein the machine learning model utilizes at least one guardrail to ensure the prediction of the likelihood of the service is accurate; determining whether the at least one policy and the data needs to be updated; upon determining the at least one policy and the data needs to be updated, updating the at least one policy and the data; reviewing the at least one policy and the data to determine all information is provided; and upon determining all the information is provided, sending the service request to the second user.

In some implementations, authenticating the first user may include providing their credentials through a computing device. The computing device may include a secure connection channeled to send the data through a communications network. Creating or selecting the service request may include browsing or querying a directory semantically related to the service request and to the data. Creating or selecting the service request may include selecting or creating a new patient record. Retrieving the data may include utilizing content-based retrieval at least one electric health record from a remote computing device. Predicting the likelihood of the service request may include matching the last one policy against the data. Predicting the likelihood of the service request may include providing alternative pathways to increase the likelihood the service request is accepted. Predicting the likelihood of the service request may include training the machine learning model with longitudinal data. Predicting the likelihood of the service request may include comparing the data with the longitudinal data to determine an outcome of the service request.

According to another aspect of the subject matter described in this disclosure, an exemplary method for reviewing an authorization request is provided. The method includes: authenticating a first user on a platform; determining whether a service request requires a decision from the first user; upon determining the service request requires the decision by the first user, retrieving at least one policy of the first user for the service request; evaluating, using a machine learning model, the at least one policy against data supplied with the service request; providing, based on the evaluation of the at least one policy against the data, a recommendation on whether to approve or deny the service request; and sending the recommendation to a second user.

In some implementations, determining whether the service request requires the decision may include providing a list of pending service requests. Providing the recommendation may include approving or denying the service request including a supporting rationale. Providing the recommendation may include, upon determining the service request is denied, providing alternate pathways to increase the likelihood the service request is accepted. Providing the recommendation may include, upon determining the service request is denied, generating a response letter explaining the denial of the service request. Evaluating the at least one policy against the data may include training the machine learning model with longitudinal data. Evaluating the at least one policy against the data may include comparing the data with the longitudinal data to determine an outcome of the service request.

According to another aspect of the subject matter described in this disclosure, an exemplary method for encoding and disambiguating information is provided. The method includes: receiving data of a first user; generating, using a machine learning model, at least one disaggregate criteria for the inclusion, exclusion, or exception of the data; and storing the disaggregate criteria into a machine comprehensible form for later retrieval.

In some implementations, the data may include at least one policy associated with at least one treatment. The data may be a human-readable document. Storing the disaggregate criteria may include disaggregating at least one policy into lists of inclusion criteria, exclusion criteria, and exception rules. Storing the disaggregate criteria may include assigning concatenating criteria with logical operators. Storing the disaggregate criteria may include storing the disaggregate criteria in a checklist.

Additional features and advantages of the present disclosure is described in, and will be apparent from, the detailed description of this disclosure.

### BRIEF DESCRIPTION OF THE DRAWINGS

The foregoing and other objects, features and advantages will be apparent from the following more particular description of the embodiments, as illustrated in the accompanying drawings in which like reference characters refer to the same parts throughout the different views. Such detailed description makes reference to the following drawings, wherein:

FIG. 1 is a high-level overview of the data architecture, illustrating the interaction between the provider and payer user interfaces, the platform's database, and the AI model for policy disambiguation and request evaluation.

FIG. 2 is a flow diagram representing the process of submitting a new prior authorization request from the provider's perspective, detailing the steps from connecting to the platform to the final transmission of the request to the payer.

FIG. 3 is a flow diagram representing the process of reviewing a submitted prior authorization request from the payer's perspective, detailing the steps from connecting to the platform to the final resolution and transmission of the decision to the provider.

FIG. 4 is a flow diagram representing the process of tracking and appealing a prior authorization request from the provider's perspective, detailing the steps of reviewing request details, adjusting evaluation criteria, generating appeal letters and submitting the appeal.

FIG. 5 illustrates the process of encoding and disambiguating payer policies, transforming the policies from a human-readable format into a machine-comprehensible form for efficient storage and retrieval in the platform's database.

FIG. 6 depicts the process of matching policy criteria with patient data from electronic health records (EHRs), mimicking the peer-to-peer review process between providers and payers to identify missing information and provide supporting evidence.

FIG. 7 illustrates the longitudinal analysis and disease progression prediction capabilities of the AI model, which compares individual patient profiles to historical cohort data to provide recommendations on request resolution and alternative care pathways.

FIG. 8 depicts the claims data analysis and prior authorization decision prediction capabilities of the AI model, which analyzes historical claims data to predict the likelihood of request approval and suggests alternative pre-approved care pathways.

FIG. 9 provides an overview of the data sources and capabilities of the platform's large language model (LLM), including the use of custom healthcare-specific embeddings, pre-training, fine-tuning, and the integration of an error detection module.

FIG. 10 is a diagram of elements of an example computing device that may be used as a client device to interact with the platform, detailing its hardware components and their interconnections.

### DETAILED DESCRIPTION

### Data Architecture

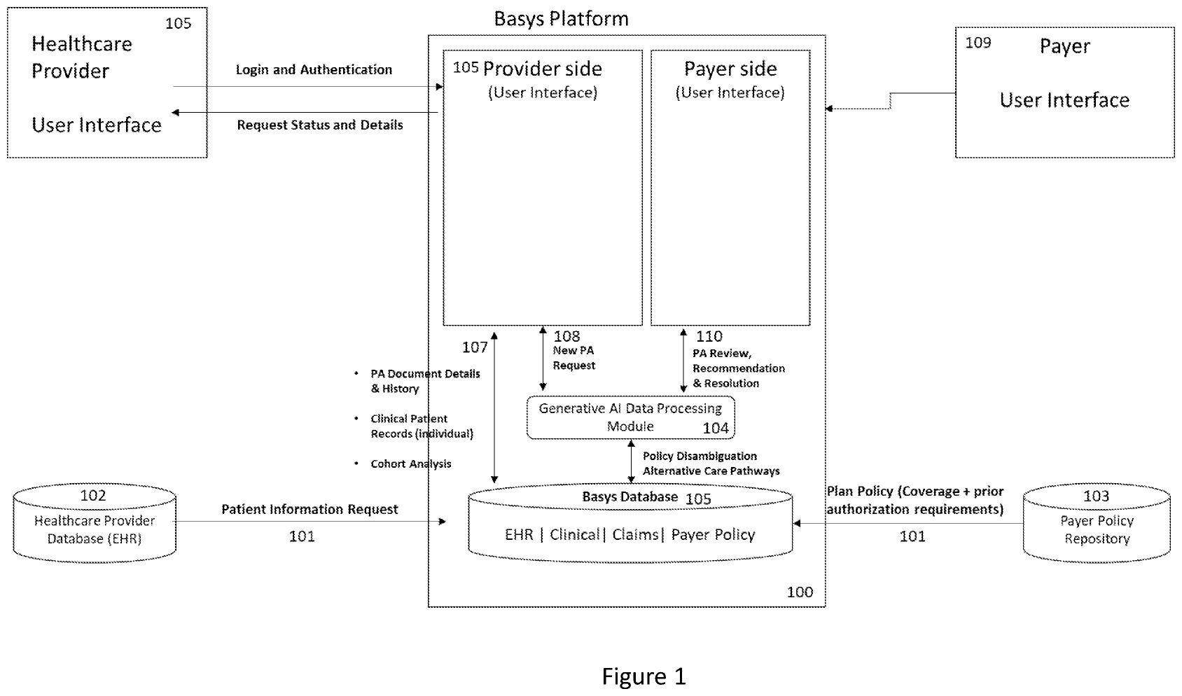

FIG. 1 shows the details of a platform along the interaction between the provider and payer user interfaces, the platform's database, and the AI model for policy disambiguation and request evaluation. The functionality of each part is explained in more detail in subsequent parts of the disclosure.

On the back end, before requests are submitted into the Basys platform (100), APIs and direct integration (101) connect the platform to the healthcare provider's Electronic Health Records (EHR) Database (102) and the payer's policy repository (103).

A generative AI model (104) disambiguates payer policies and encodes them as an algorithm that takes EHR data as inputs and returns lists of policy criteria matched with patient data. These disambiguated policies can either have persistent storage state status in the platform's database (105), or the engine can create the policy evaluations on the fly when requested by the engine. This ensures that always the latest, updated policies are pulled from the Payer Policy Repository and used for the disambiguation process.

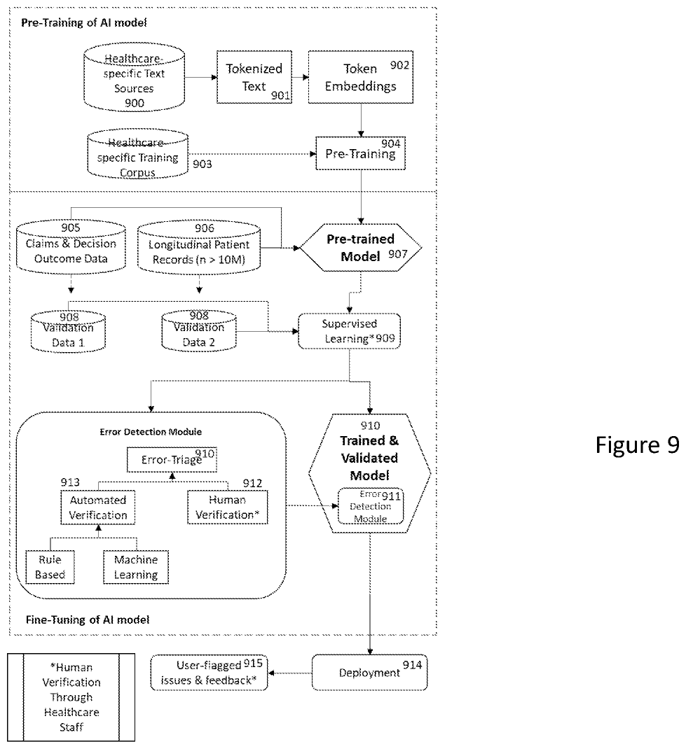

The generative AI model was fine-tuned with clinical oversight and trained with specialized embeddings. The model also incorporates guardrails to prevent unsafe or biased outputs, such as avoiding the generation of protected health information and monitoring for potential errors or inconsistencies. The details of this are discussed later in FIG. 9.

On the front end, when providers login and begin interacting with the provider-side user interface (106), the interface makes calls to the basys.ai database that are mediated by the platform. If the provider is checking the status of existing service/prior authorization (PA) requests, this data is automatically retrieved from the database without use of AI (107).

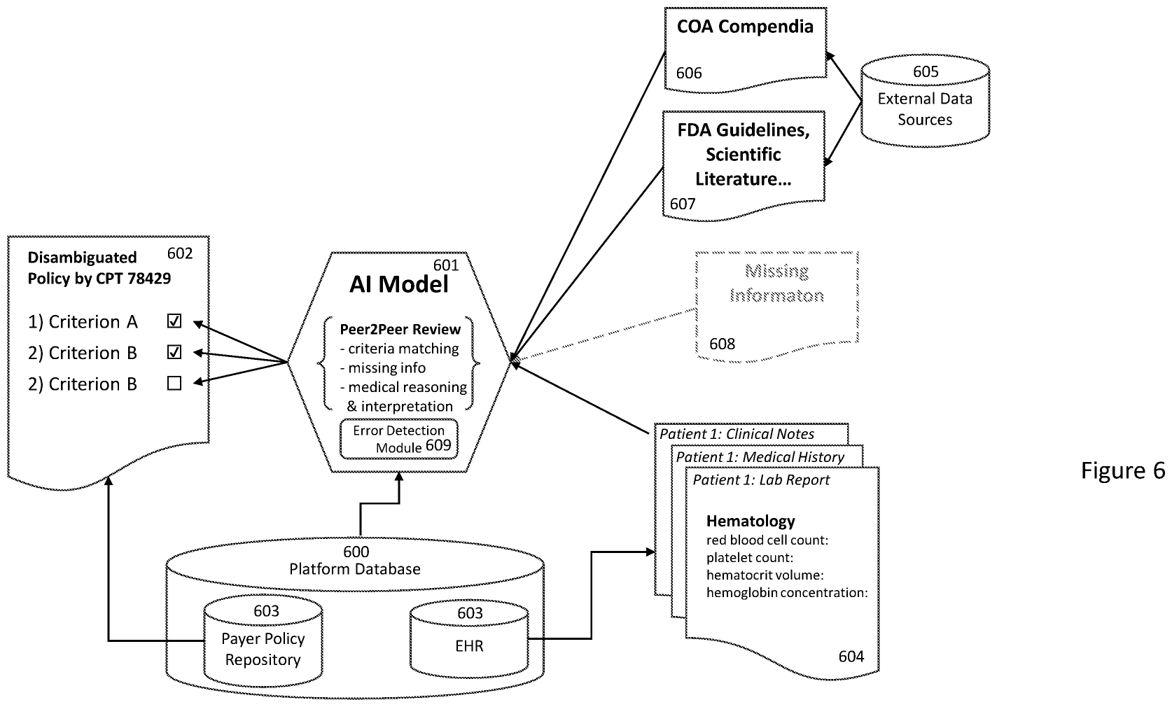

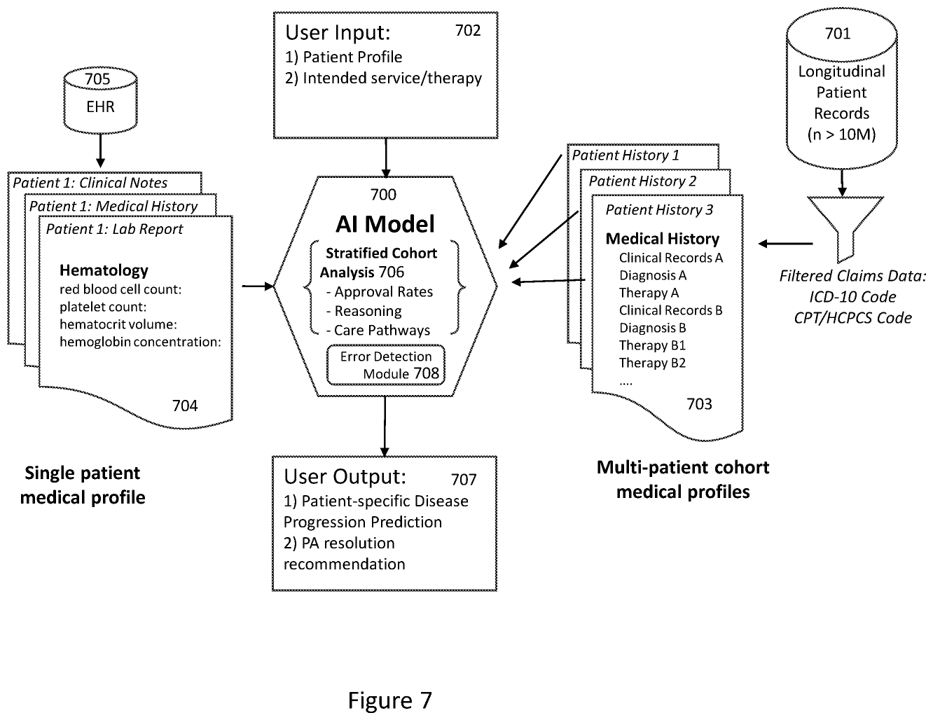

If the provider is generating a new PA request to submit on behalf of a patient, the engine automatically matches EHR data to policy requirements in request forms (108). The details of this process are explained in FIG. 6. Upon submission of the form, the provider-inputted data is sent to the platform's database and passed through the AI model which conducts longitudinal analysis to provide the payer (109) with a recommendation on the PA decision (110). The details of this process are explained in FIG. 7.

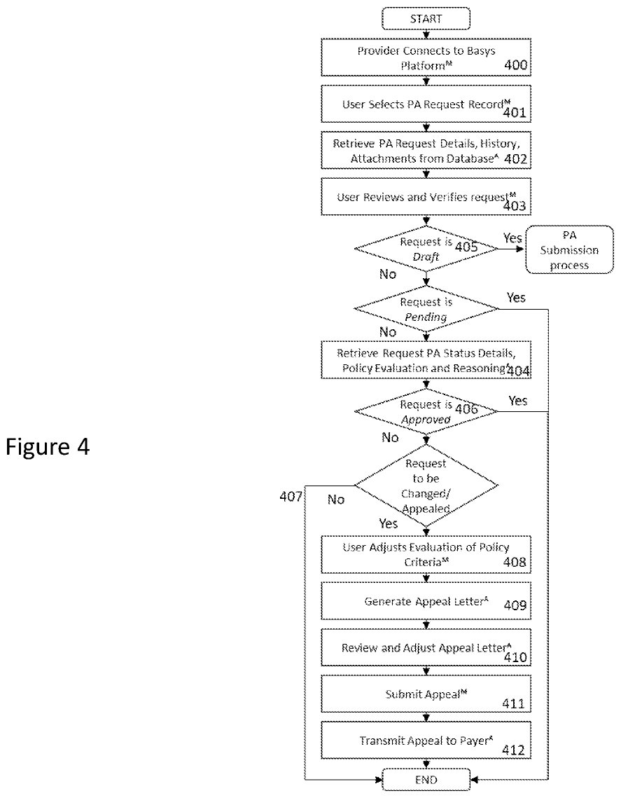

After receiving a decision from the payer (approval or denial), the model updates the existing PA request record in the database. The provider may then request an appeal, at which point the engine that conducts longitudinal and cohort analyses presents relevant pre-approved alternative care pathways, help draft the appeal letter, and provide a final recommendation to the payer. The details of this process are explained in FIG. 4.

### Service Request Submission Process Flow (Provider)

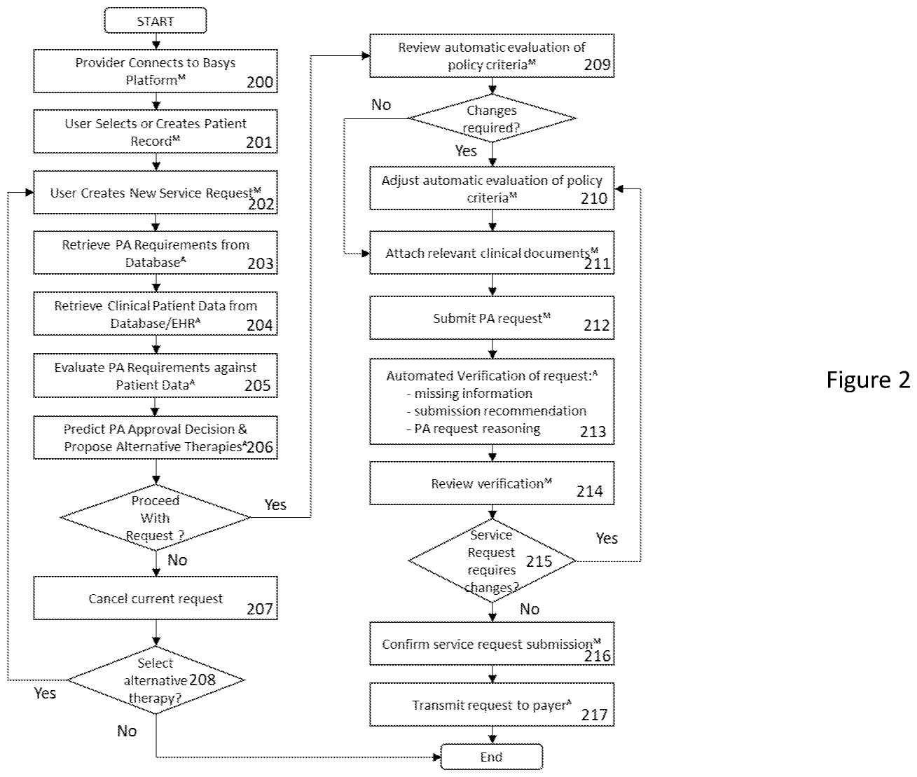

FIG. 2 describes the process flow of submitting prior authorization requests through the platform. Steps that are triggered by or that require manual input from the user are denoted with a superscript “M”. Steps that are launched or processed automatically by the platform are denoted with a superscript “^{A}”.

A healthcare provider can connect with the platform (200) by providing their credentials through their device. The authentication and data exchange process are handled via a secure connection channeled through a communications network.

Once the connection is established, the provider my browse or query the patient directory and either select or create a new patient record (201). After selecting or creating a patient record in the user interface, the provider may create a new request for a particular therapy service for the selected patient (202).

In the context of this technology, “therapy” refers to any diagnostic or therapeutic procedure, treatment, drug, or other medical intervention that is intended to address the patient's health condition. This therapy may be referred to by its description or any codified system such as but not limited to the Current Procedural Terminology (CPT), the Healthcare Common Procedure Coding System (HCPCS), National Drug Code (NDC) and billing codes.

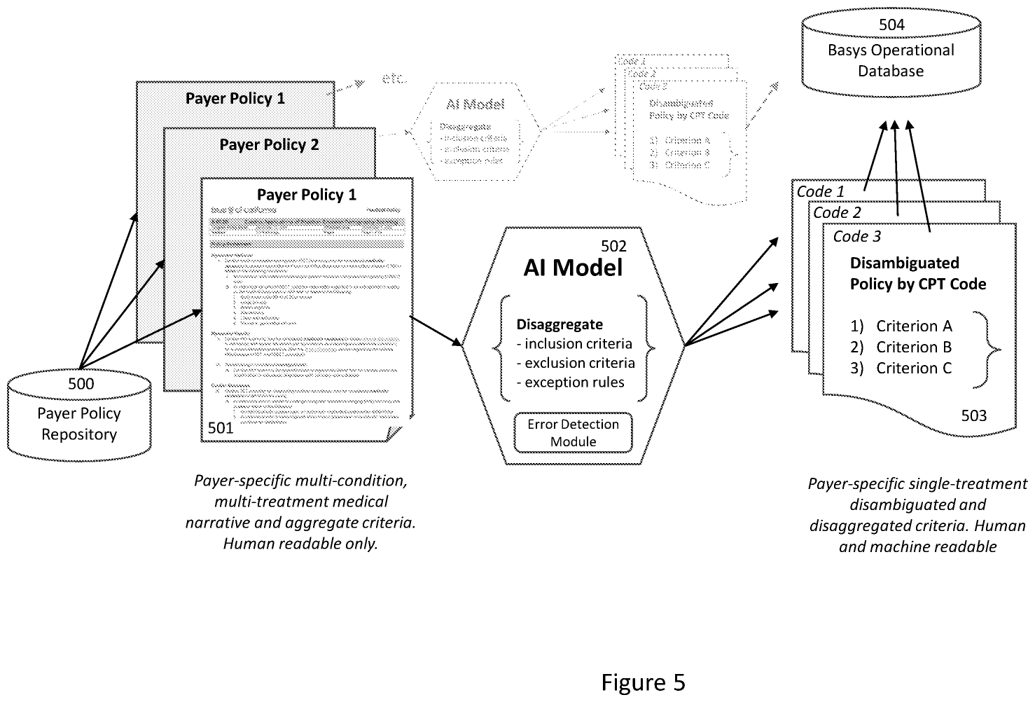

The platform then retrieves the relevant requirements for the selected service request according to the policy/policies of the payer (203). The methodology of encoding payer policies and deriving specific requirements is explained in detail in FIG. 5.

Subsequently, the platform retrieves the relevant patient data from the EHR system (204) that is pertinent to evaluating the policy requirements established in the previous step or providing evidence to support this evaluation.

In step (205), each policy criterion is matched by the engine against the clinical data. The outcome of this evaluation is a binary result marked as either met or unmet. The engine also includes a summary of the source information meeting the criterion as it was found in the patient's EHR data. The methodology of matching policy criteria and clinical patient data is explained in detail in FIG. 6.

Based on this evaluation and comparing it to historic data, the engine predicts the likelihood of this service request being denied or approved. Moreover, the engine will flag potential preapproved care pathways to the provider if they show a better approval prediction (206).

The provider also has the option to terminate the request submission process at any point (207). If they decide to cancel the current request, they can decide to proceed with a different care pathway towards submission/approval and repeat the abovementioned steps with the newly selected treatment (208).

Once the provider has reviewed the information input provided by the engine (209), they may desire to make changes (210) and upload additional documentation where necessary (211). Such additional information may also include information that is specific to a therapy such as but not limited to the frequency and number of interventions, doses, or therapy sessions.

After completing the above steps, the provider may now proceed to submitting the service request (212). The platform conducts a final automated evaluation of the request and flags missing information to the provider that may result in denial (213) and the engine also provides the expected resolution of the request along with a justification.

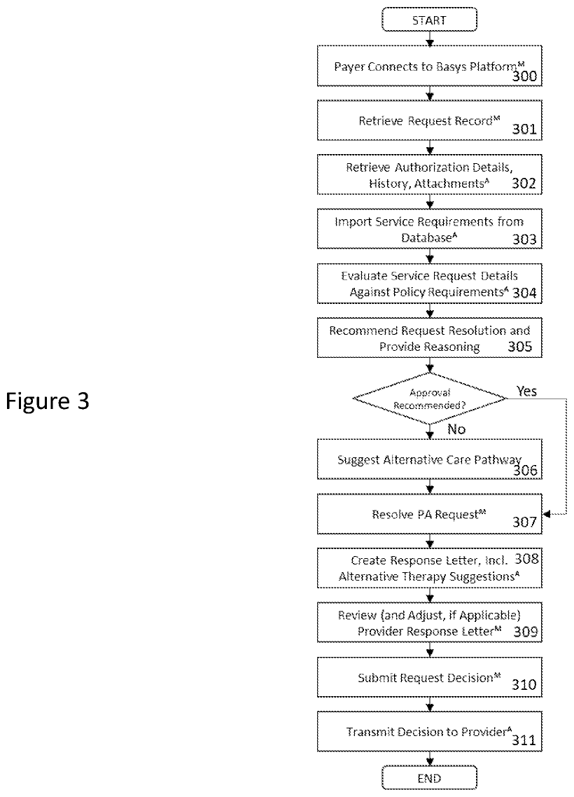

After the final review from the provider (214), they may return to earlier steps to adjust the request evaluation or upload additional documentation (215). Alternatively, they may now confirm request submission (216). Upon confirmed submission (217), the services request is transmitted to the payer and the workflow of the payer side of the platform (described in FIG. 3) is initiated.

The abovementioned process is identical for when an existing service request is to be appealed, but additional information is added: The engine analyzes the payer's stated reason(s) for the original denial and notifies the provider of the specific criteria or requirements that led to the denial. The provider is then prompted to submit additional supporting documentation and written rationale explaining why the request is still medically necessary despite the identified deficiencies. The service request will now also contain the full case history including all previous requests, prior denial letters and supporting documentation added as a response from the provider.

Moreover, the abovementioned process was illustrated with the scenario of a single therapy. However, the therapy may consist of may consist of unlimited number of therapy combinations encompassing different related or unrelated therapy selections. All previously and subsequently described steps will follow the same logic even if the service request encompasses several therapy modules or codes.

In one implementation, the abovementioned process is extended through the connection of additional services to cover functionality that is specific to the provider/health system or the payer that the provider is sending the service request to. Those additional services depend on the availability of data sources and connections and may include but are not limited to the verification of and adjustment of the request to the providing site (e.g. in-patient, out-patient, at-home services), the status of the provider (e.g. in- or out-of-network) and the benefit/coverage of the particular patient based on their individual health plan.

### Service Request Review Process Flow (Payer)

FIG. 3 visualizes the workflow for the review of service requests on the payer side. Steps that are triggered by or that require manual input from the user are denoted with a superscript “^{M}”. Steps that are launched or processed automatically by the platform are denoted with a superscript “^{A}”.

Similarly to a provider user, a payer can connect with the platform (300) by providing their credentials through their device. The authentication and data exchange process are handled via a secure connection channeled through a communications network.

Once the connection is established, a payer can see a list of pending provider PA requests (300). From this list, any record can be selected (301) to view the case details, history and attachments (302) of any particular service request.

If the selected service request requires a decision from the payer, the platform retrieves the policy requirements for this service request (303) from its database. The methodology of encoding payer policies and deriving specific requirements is explained in detail in FIG. 5.

Analogous to the process on the provider side described above, the platform evaluates the policy requirements against the medical details supplied with the service request (304). This evaluation is then translated into recommendation on whether to approve or deny the request including a supporting rationale (305). The methodology of matching policy criteria and clinical patient data is explained in detail in FIG. 6.

If the engine does not recommend the approval of the service request, the engine may propose any care pathway alternatives that could be available to the patient based on the medical profile and evidence submitted (306). The methodology of identifying alternative care pathways is explained in detail in FIG. 7.

Based on this information, the payer may take a decision to resolve (approve or deny) or dispute (request more information or request external medical review) the service request (307). If they deny the request, the engine generates a response letter explaining the rationale for the denial (308). The payer may then decide to include the proposed letter with their decision or edit its content prior to resolving the request (309).

Once the payer confirms the submission of the request decision (310), it is transmitted back to the provider (311).

The abovementioned process is identical for reviewing a service request appeal, but additional information is added: When the payer reviews the appeal, they are presented with this extracted summary highlighting just the pertinent new evidence related to the denial, allowing for an efficient peer-to-peer review. Evidence extraction and summarization utilizes machine learning to identify contextual patterns between documentation and specific policy requirements/denial reasons.

This streamlined appeals process reduces back-and-forth between providers and payers by guiding providers on what evidence to provide and giving payers a clear synopsis of how the new information addresses the original denial reasons. It mimics and improves the peer-to-peer review process in the case of service request denials/appeals, analogously to what is explained in FIG. 6.

### Service Request Tracking and Appeal Process (Provider)

FIG. 4 describes the tracking and appeal process flow on the provider side. Steps that are triggered by or that require manual input from the user are denoted with a superscript “^{M}”. Steps that are launched or processed automatically by the platform are denoted with a superscript “^{A}”.

A healthcare provider can connect with the platform by providing their credentials through their device (400). The authentication and data exchange process are handled via a secure connection channeled through a communications network.

Once the connection is established, the provider can search, browse or query the list service requests previously generated and see them through to a final decision.

For services requests that are in “pending” status, the provider can review the information submitted but can not effectuate any changes. For services requests that are in “draft” status, the provider may continue the submission process flow as described in FIG. 2 (405).

For services requests that are in status “approved” (406), the process is completed, and no changes can be made. Same is the case for services requests that are in status “denied” and which the provider does not decide to dispute.

For services requests that are in status “request for more information” or if the provider decides to appeal a “denied” request (407), the provider may review and adjust the policy requirement evaluation result (408) (see FIG. 2) in alignment with the payer's feedback.

The provider may also review and adjust the appeal letter (409) explaining the medical necessity of treatment and presenting the required evidence that is automatically generated by the engine (410).

After completing the appeal process on the payer side, the user may submit the appeal (411). This will transmit the information to the payer, who may now review and the updated information (412). This process is identical to the flow explained in FIG. 3.

### Policy Encoding and Disambiguation

FIG. 5 describes the process of encoding and disambiguating medical policies of payers. The policies may carry names such as but not restricted to medical policy, coverage policy or plan policy and determine the coverage criteria of members of a specific health plan as well as medical or administrative guidelines.

The platform receives these payer policies as human-readable documents directly from the repositories of health plans (500). This might be but is not restricted to online portals and documents hosted on websites. Every payer may have one or more policies pertaining to one or several medical conditions. Their policies may cover one or more and typically cover multiple possible treatments for any medical condition. (501)

Every policy is processed by the AI model to disaggregate criteria for the inclusion, exclusion, or exception of members from coverage for any given treatment or several treatments. (502)

Subsequently, the encoded and disambiguated criteria are stored in the form of simplified checklists of criteria that are machine comprehensible, as opposed to their original textual format, which are only human comprehensible. (503)

The process of disambiguation involves disaggregating the policies into lists of inclusion criteria, exclusion criteria, and exception rules as well as assigning concatenating criteria with logical operators (such as AND/OR). (504) Thus, any possible inclusion or exclusion logic provided by the original policy can be derived and stored in the database. The criteria- and rulesets are organized and stored by treatment, whereas policies typically cover multiple treatment. The significance of this encoding and storage process is that it allows for the engine to retrieve policy criteria instantaneously and when matching and evaluation policy requirements with EHR data. This process is explained in FIG. 6.

The process illustrated above is repeated for every policy found in the policy repository and is repeated for every update of each policy.

### Service Request Criteria Matching and Peer-to-Peer Review

FIG. 6 describes the process of matching disambiguated policy criteria with form the EHR patient data.

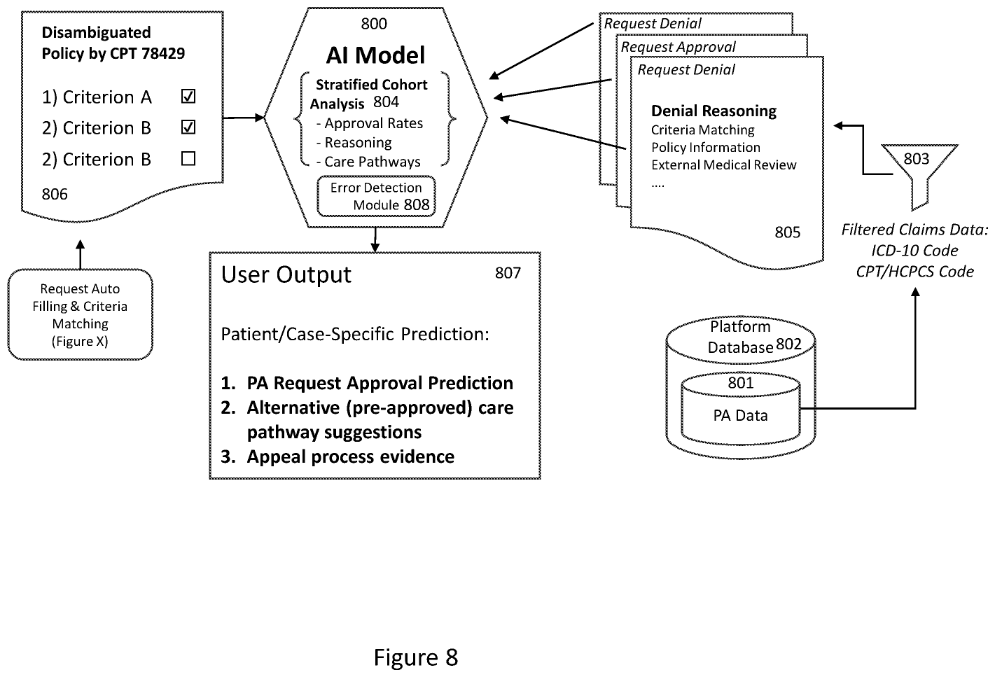

When a provider initiates a new prior authorization request (as described in FIG. 2), selects a treatment for a given patient, and confirms that they wish to proceed with this treatment (instead of a potential alternative an alternative treatment that may be proposed as explained in FIG. 8) the platform retrieves the related payer policy criteria and automatically matches them to the patient's EHR data.

The disambiguated payer policy criteria are stored in the platform's database (600). This is used by the engine (601) to generate a list of coverage criteria including any potential inclusion decision logic such as AND, OR, AT LEAST TWO OF . . . ” (602).

The engine pre-fills patient information by matching data points from the EHR (603) to each policy criterion. To this end, the engine reads and retrieves individual records including clinical notes, medical history, lab reports, and imaging results from the EHR data (604). This data is read independently of the file format and structure of the underlying documents. As supporting evidence, the engine includes both a summary of each data point and an attachment with the full document. In addition to matching relevant documentation that is called for by each policy criterion, the platform includes medical reasoning for why a given treatment is necessary.

In addition to sourcing this reasoning from a comparison of the payer policies to the EHR data, additional information can be incorporated while providing attribution to external sources (605) such as compendia (606), FDA guidelines and scientific literature (607) to provide supporting evidence on cases such as off-label medication use.

This information is either pre-filled into the prior authorization form or used when automatically generating a request or appeal letter. The engine also attaches similar services request cases and predictions of patient outcomes on the selected care pathway given their disease progression. The details of this approach are described in FIG. 7. Providers are ultimately responsible for deciding which information to retain, and they are able to edit, delete, and add whatever information and documentation they deem relevant. The platform merely expedites the process by providing information that is relevant given the patient's medical history and the payer policy requirements.

This process is intended to mimic the peer-to-peer review that occurs between payers and providers. During the conventional (manual) service request process this exchange is typically a time-consuming communication handled via phone, fax and written exchange of designated focal points who discuss payer policies and clinical patient information.

The platform's engine on the other hand aligns patient medical history with payer policies and flags to providers where information is missing (608) already before they submit the request. This constitutes a shortcut of the traditional feedback loop between provider and payers using email/phone/fax communication that is typically evolving over multiple days or weeks and associated with adverse outcomes like request denials and delayed therapy for patient. The platform replaces this with a more transparent process and instantaneous information to help guide decisions.

The engine includes a module to prevent and detect errors, with built-in guardrails (609), which is explained in detail in FIG. 9.

### Longitudinal Analysis and Disease Progression Prediction

FIG. 7 visualizes the component of the engine that analyses longitudinal clinical cohort data and compares it to individual patient profiles to predict disease progression features.

In order to provide assistance to payers when by recommending service request resolutions (“approve”, “deny”, “request additional information” or “request external review”) the engine is running a machine learning algorithm (700) that is trained on over 10 million longitudinal medical records (701). This enables the platform to predict a patient's disease progression and outcomes given a specific selected or requested care pathway (702).

The engine now conducts a longitudinal analysis, comparing the medical history of patients in its training data that underwent similar care pathways (703) to that which is recommended for the target patient and the data contained in the target patient's profile (704). The patient profile data is retrieved from the EHR system (705) once a target patient is selected. The engine accounts for the similarity of patients in the training data to the target patient across a range of variables, including diagnoses, comorbidities, prior conservative therapies, demographics, and other confounding variables using stratified cohort analysis (706). Based on several KPIs, including 90-day readmission rates and care plan adherence, this algorithm gives a recommendation to payers on how to resolve (“approve” or “deny”) or dispute (“request additional information” or “request external review”) a service request (707).

While the payers retain agency over all decisions taken on service requests the data-driven recommendation on predicted patient outcomes reduces administrative burden, supports the decision-making process on the payer side and aligns incentives with patient needs.

The Longitudinal analysis also applies to recommendations for payers in deciding on appealed service request decisions. From a technical perspective, the review process is analogous to reviewing new service requests. The machine learning algorithm effectively maps a decision tree of approvals, denials, and alternative care pathways to patient outcomes as illustrated in the following example process.

In order to receive coverage for a certain anti-obesity drug, a patient must show a BMI above 40 kg/m². The patient meets this criterion, and their request is initially approved. However, due to successful usage of the drug, their BMI drops to 37 kg/m². The patient is no longer eligible for the drug and discontinues usage. Then, their BMI increases to 39 kg/m². Even though the patient has not yet reached the required BMI threshold, they should not be denied as they are experiencing a rebound in symptoms. A longitudinal analysis could provide support to prevent negative outcomes in the case of an abandoned therapy with this drug. Therefore, the engine recommends that the payer approve the drug.

The engine includes a module (708) to prevent and detect errors, with AI guardrails to ensure clinically safe and accurate outputs, which is explained in detail in FIG. 9.

### Service Request Cohort Data Analysis

FIG. 8 visualizes the engine's feature to analyze historical claims data in order to provide predictions on service request approval decisions as well as proposing alternative care pathway suggestions.

The platform's AI engine (800) has been trained with historic service request data which allows it to conduct cohort analyses of similar cases to predict service request decision outcomes for specific therapies. Specifically, the payer's prior affirmative decisions on similar cases serve as precedent for the validity of a possible appeal when a provider is faced with a denial of their prior authorization request.

When querying a specific treatment and code, the engine retrieves historic service request data (801) from its database (802) and determines which requests were submitted for the same or related treatments/codes (803).

The engine conducts a stratified cohort analysis (804) comparing the care pathways of the patients (805) underlying the historic request data their decision statuses, and the reasoning given for each decision made by the payer. Then, it compares the target patient's profile and the degree to which their EHR data matches the disambiguated payer policies (806) to these other request data. This provides evidence in the appeal letter to substantiate the provider's request and is factored into the final recommendation given to the payer, along with the result of the longitudinal analysis described in FIG. 7.

Thus, the engine can recognize patterns of criteria-EHR combinations that are likely resulting in denials, for different service types and will then notify the provider which specific criteria contribute to the likely denial prediction. The user has thus the opportunity to preemptively submit additional supporting documentation and written rationale explaining why the request is still deemed medically necessary to support the patient case. This supporting evidence is attached to the prior authorization submission, allowing the payer to review the provider's perspective and justification upfront when evaluating the request initially, rather than going through a denial and appeal cycle. This approach helps to streamline the peer-to-peer review process in the case of service request denials/appeals, as explained in detail in FIG. 6.

Upon completion of this process, this aggregated information is made available to the person reviewing the request or its decision and may also be used to aid in drafting appeal letters and giving recommendations to payers for decisions on newly submitted or appealed service requested. Moreover, when a payer denies a PA request, providers can view the updated status and request an appeal. When requesting an appeal, the platform presents providers with alternative care pathways that are pre-approved as user output (807) (See FIG. 7).

The purpose of this feature is that if providers are presented with several treatments and deem one to be equally efficacious as their original choice but are informed that the second option will certainly be covered by insurance and can receive instant approval, they may take this into account when deciding on treatment. However, they are not being advised on the medical necessity of a given treatment, nor are they being prevented from requesting treatments that are typically not covered. Providers are ultimately the arbiters of patient care, and basys.ai supports them both in selecting treatments that will be reimbursed and in evidencing medical necessity for treatments that health plans may not typically cover. Once a provider confirms their preferred treatment, the platform generates an appeal letter based on EHR data and clinical literature, as discussed elsewhere in this document.

The engine includes a module (808) to prevent and detect errors, incorporating robust guardrails, which is explained in detail in FIG. 9.

### AI Model Data Sources and Capabilities

FIG. 9 provides an overview on the data sources and features of the platform's large language model (LLM).

The basys.ai generative AI models are trained with custom language embeddings, enhancing their accuracy across specific medical specialties. The creation of these embeddings entails the definition of medical vocabulary as vectors, enabling precise encoding of payer policies into the basys.ai database through natural language processing (NLP). These embeddings have been built specifically for various disease areas, including musculoskeletal (MSK)/surgery, cardiology, and immunology.

Once the embeddings (902) are established a healthcare specific training corpus was used for the pre-training phase of the model (904). The pre-trained model (907) is then provided with historical data on both service requests including decision outcomes (905) and longitudinal data (906) from containing clinical records of more than 10 million patients. From both sources data is split to serve as validation data (908) later on. A supervised learning approach (909) is used to finetune the model based with PA and clinical data and validated against the previously split validation data.

The LLM is now finetuned and completed by an error detection module (911). This module triages potential errors through either automated verification (913) or human verification (912). The former methodology uses both rule-based detection mechanism and AI-based algorithms to detect errors in the primary AI-engine's output.

After deployment (914), an additional functionality allows any user to manually flag issues (915) and raise potential errors of the engine for manual review. That means that the flagged output is reported to the same previously mentioned medical staff who are responsible for verifying the information.

Any error that is found is eventually confirmed through human model supervisors. The errors are logged, if required examined with additional investigation and handed over to the engineering team to make the necessary adjustments at the fine-tuning step of the next model iteration. By implementing penalty functions and algorithms that monitor and review output (as explained above), the basys.ai models self-train and converge on reducing errors over time.

They review and categorize errors made by the engine both during training and development. These categories include but are not restricted to hallucinating information, misunderstanding medical vocabulary, and incorrectly encoding logical expressions.

The human model supervisors consist of Medical doctors (MDs) who oversee the entire development process of the model and who are responsible to guarantee clinical accuracy of all generated output.

In summary, the error detection module ensures that the AI model incorporates a set of guardrails to ensure safe, unbiased, and clinically accurate outputs. These guardrails include techniques to prevent the generation of protected health information, detect potential inconsistencies or inaccuracies in generated content, and monitor for biased language or recommendations. This approach ensures that any output or reviews generated by the AI model are free from discrimination based on gender, race, age, disability, or other characteristics of individuals or population groups.

The guardrails are continuously refined based on feedback from the human model supervisors and ongoing testing and validation of the model's performance. The totality of these measures improves the transparency and explainability of the AI model output.

### Client Computing Devices

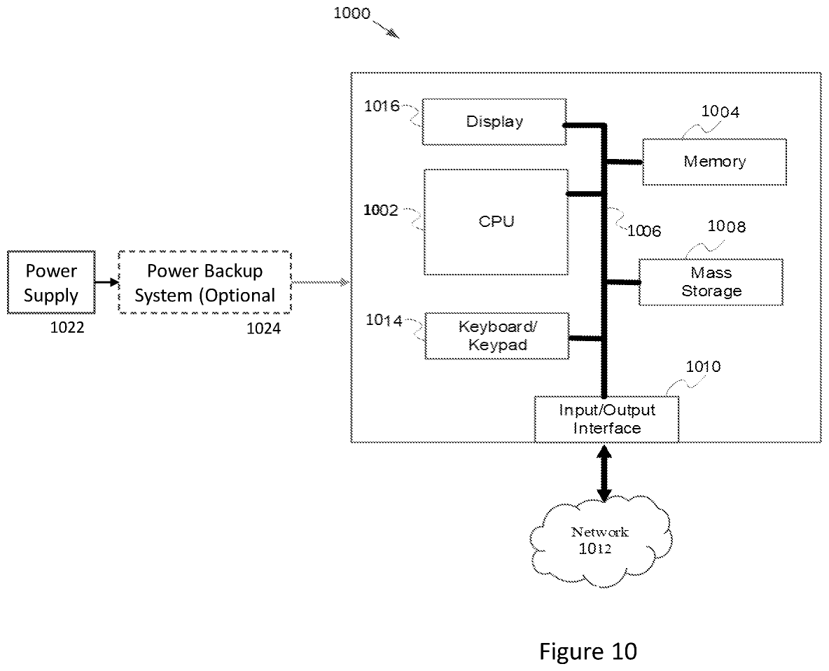

FIG. 10 shows a diagram of an exemplary computer system (1000) arranged to perform functions associated with platform (100) of FIG. 1. The computer system (1000) may be implemented as a virtual machine or a physical machine. The exemplary computer system (1000) includes a central processing unit (CPU) (1002), a memory (1004), and an interconnect bus (1006). The CPU (1002) may include a single microprocessor or a plurality of microprocessors or special purpose processors for configuring computer system (1000) as a multi-processor system. The memory 1004 illustratively includes a main memory and a read only memory. The computer (1000) also includes the mass storage device (1008) having, for example, various disk drives, tape drives, etc. The memory 1004 also includes dynamic random-access memory (DRAM) and high-speed cache memory. In operation, memory (1004) stores at least portions of instructions and data for execution by the CPU (1002). The memory (1004) may also contain computing elements, such as Deep In-Memory Architectures (DIMA), wherein data is sent to memory and a function of the data (e.g., matrix vector multiplication) is read out by the CPU (1002).

The mass storage (1008) may include one or more magnetic disk, optical disk drives, and/or solid-state memories, for storing data and instructions for use by the CPU (1002). At least one component of the mass storage system (1008), preferably in the form of a non-volatile disk drive, solid state, or tape drive, stores a database used for processing data and controlling functions associated with receiving user inputs and/or display data associated with an object trajectory prediction system such as system (1000). The mass storage system (1008) may also include one or more drives for various portable media, such as a floppy disk, flash drive, a compact disc read only memory (CD-ROM, DVD, CD-RW, and variants), memory stick, or an integrated circuit non-volatile memory adapter (i.e. PC-MCIA adapter) to input and output data and code to and from the computer system (1000).

The computer system (1000) may also include one or more input/output interfaces for communications, shown by way of example, as interface (1010) and/or a transceiver for data communications via the network (1012). The data interface (1010) may be a modem, an Ethernet card or any other suitable data communications device. To provide the functions of a processor according to FIG. 10, the data interface (1010) may provide a relatively high-speed link to a network (1012), such as an intranet, internet, or the Internet, either directly or through another external interface. The communication link to the network (1012) may be, for example, optical, wired, or wireless (e.g., via satellite or cellular network). The computer system (1000) may also connect via the data interface (1010) and network (1012) to at least one other computer system to perform remote or distributed object trajectory prediction operations. Alternatively, the computer system (1000) may include a mainframe or other type of host computer system capable of Web-based communications via the network (1012). The computer system (1000) may include software for operating a network application such as a web server and/or web client.

The computer system (1000) may also include suitable input/output ports, that may interface with a portable data storage device, or use the interconnect bus (1006) for interconnection with a local display (1016), computer mouse, and keyboard (1014) or the like serving as a local user interface for programming and/or data retrieval purposes. A mouse may enable a user to position a pointer over a selectable icon and/or button on display (1016) to enable the user to make selections and/or configure an object trajectory prediction system to implement a tracking model and/or display selected graphical or other data associated with tracking an object. The display (1016) may include a touch screen capability to enable users to interface with the system (1000) by touching portions of the surface of the display (1016). Server operations personnel may interact with the system (1000) for controlling and/or programming the system from remote terminal devices via the network (1012).

The computer system (1000) may run a variety of application programs and store associated data in a database of mass storage system (1008). One or more such applications (1020) may include an object trajectory prediction system according to FIG. 10. The components contained in the computer system (1000) may enable the computer system to be used as a server, workstation, personal computer, network terminal, mobile computing device, mobile telephone, System on a Chip (SoC), and the like. As discussed above, the computer system (1000) may include one or more applications such as system (1000). The system (1000) may include software and/or hardware that implements a web server application. The web server application may include software such as HTML, XML, WML, SGML, PHP (Hypertext Preprocessor), CGI, and like languages.

The foregoing features of the disclosure may be realized as a software component operating in the computer system (1000) where the operating system includes Unix workstation, a Windows workstation, a LINUX workstation, or other type of workstation. Other operating systems (1018) may be employed such as, without limitation, Windows, MAC OS, and LINUX. In some aspects, the software can optionally be implemented as a C language computer program, or a computer program written in any high level language including, without limitation, MATLAB, JavaScript, Java, CSS, Python, Keras, TensorFlow, PHP, Ruby, C++, C, Shell, C#, Objective-C, Go, R, TeX, VimL, Perl, Scala, CoffeeScript, Emacs Lisp, Swift, Fortran, Visual BASIC, HDL, VHDL, and/or one or more versions of Verilog. Certain script-based programs may be employed such as XML, WML, PHP, and so on. The system 1000 may use a digital signal processor (DSP).

As stated previously, the mass storage (1008) may include a database. The database may be any suitable database system, including the commercially available or open-source products, such as, but not limited to, Microsoft Access, Sybase, SQL Server, MongoDB, SQLite. The database can be implemented as a local or distributed database system. The database may be supported by any suitable persistent data memory, such as a hard disk drive, RAID system, tape drive system, floppy diskette, or any other suitable system. The system (1000) may include a database that is integrated with the system, however, it will be understood that, in other implementations, the database and mass storage (1008) can be an external element. The database may include object trajectory and/or flight path files, filter modules, sensor modules, and one or more flight path models and/or algorithms associated with system (1000).

In certain implementations, the system (1000) may include an Internet browser program and/or be configured to operate as a web server. In some configurations, the client and/or web server may be configured to recognize and interpret various network protocols that may be used by a client or server program. Commonly used protocols include Hypertext Transfer Protocol (HTTP), File Transfer Protocol (FTP), Telnet, and Secure Sockets Layer (SSL), and Transport Layer Security (TLS), for example. However, new protocols and revisions of existing protocols may be frequently introduced. Thus, in order to support a new or revised protocol, a new revision of the server and/or client application may be continuously developed and released.

In one implementation, the platform (100) and/or system (1000) includes a networked-based, e.g., Internet-based, application that may be configured and run on any combination of the other components of system (100) and/or system (1000). The computer system (1000) may include a web server running a Web 2.0 application or the like. Web applications running on system (1000) may use server-side dynamic content generation mechanisms such, without limitation, Java servlets, CGI, PHP, or ASP. In certain embodiments, mashed content may be generated by a web browser running, for example, client-side scripting including, without limitation, JavaScript and/or applets on a wireless device.

In certain implementations, platform (100) and/or system (1000) may include applications that employ Verilog HDL, VHDL, asynchronous JavaScript+XML (Ajax) and like technologies that use asynchronous loading and content presentation techniques. These techniques may include, without limitation, XHTML and CSS for style presentation, document object model (DOM) API exposed by a web browser, asynchronous data exchange of XML data, and web browser side scripting, e.g., JavaScript. Certain web-based applications and services may utilize web protocols including, without limitation, the services-orientated access protocol (SOAP) and representational state transfer (REST). REST may utilize HTTP with XML.

The platform (100) and/or computer system (1000) may also provide enhanced security and data encryption. Enhanced security may include access control, biometric authentication, cryptographic authentication, message integrity checking, encryption, digital rights management services, and/or other like security services. The security may include protocols such as IPSEC and IKE. The encryption may include, without limitation, DES, 3DES, AES, RSA, ECC, and any like public key or private key based schemes.

Additionally, the device is equipped with a power supply (1022) designed to provide the necessary electrical energy for its operation. This power supply can vary in form, depending on the device's specific requirements and the nature of its use, ensuring consistent and reliable performance under a range of conditions.

Moreover, there is provision for an optional power backup unit (1024) within the device's architecture. This auxiliary power source is intended to maintain the device's operation during instances of primary power supply failure or when operating in environments where power fluctuations are common. The inclusion of this optional power backup unit enhances the device's reliability and usability, providing users with an uninterrupted experience even in the face of power instability. Such a backup power solution could take various forms, including, but not limited to, battery packs, uninterruptible power supplies (UPS), or other energy storage technologies, each selected based on the device's operational requirements and the anticipated scenarios of use. This foresight in design ensures that the device remains functional and accessible, preserving data integrity and continuing critical operations without disruption, thereby significantly benefiting the user's experience and the device's overall efficacy.

Finally, the above descriptions of the implementations of the present disclosure have been presented for the purposes of illustration and description. It is not intended to be exhaustive or to limit the present disclosure to the precise form disclosed. Many modifications and variations are possible in light of the above teaching. It is intended that the scope of the present disclosure be limited not by this detailed description, but rather by the claims of this application. As will be understood by those familiar with the art, the present disclosure may be embodied in other specific forms without departing from the spirit or essential characteristics thereof. Accordingly, the present disclosure is intended to be illustrative, but not limiting, of the scope of the present disclosure, which is set forth in the following claims.

What is claimed is:

1. A method for submitting an authorization request, the method comprising:
authenticating a first user on the platform;
creating or selecting, by the first user, a service request for a second user;
retrieving requirements for the service request according to at least one policy of a second user;
retrieving data that is pertinent to evaluating the at least one policy;
predicting, using a machine learning model, the likelihood of the service request being denied or approved, wherein the machine learning model utilizes at least one guardrail to ensure the prediction of the likelihood of the service is accurate;
determining whether the at least one policy and the data needs to be updated;
upon determining the at least one policy and the data needs to be updated, updating the at least one policy and the data;
reviewing the at least one policy and the data to determine all information is provided; and
upon determining all the information is provided, sending the service request to the second user.

2. The method claim 1, wherein authenticating the first user comprises providing their credentials through a computing device.

3. The method claim 1, wherein the computing device comprises a secure connection channeled to send the data through a communications network.

4. The method of claim 1, wherein creating or selecting the service request comprises browsing or querying a directory semantically related to the service request and to the data.

5. The method of claim 1, wherein creating or selecting the service request comprises selecting or creating a new patient record.

6. The method of claim 1, wherein retrieving the data comprises utilizing content-based retrieval at least one electric health record from a remote computing device.

7. The method of claim 1, wherein predicting the likelihood of the service request comprises matching the last one policy against the data.

8. The method of claim 1, wherein predicting the likelihood of the service request comprises providing alternative pathways to increase the likelihood the service request is accepted.

9. The method of claim 1, wherein predicting the likelihood of the service request comprises training the machine learning model with longitudinal data.

10. The method of claim 1, wherein predicting the likelihood of the service request comprises comparing the data with the longitudinal data to determine an outcome of the service request.

11. A method for reviewing an authorization request, the method comprising:
authenticating a first user on a platform;
determining whether a service request requires a decision from the first user;
upon determining the service request requires the decision by the first user, retrieving at least one policy of the first user for the service request;
evaluating, using a machine learning model, the at least one policy against data supplied with the service request;
providing, based on the evaluation of the at least one policy against the data, a recommendation on whether to approve or deny the service request; and
sending the recommendation to a second user.

12. The method claim 11, wherein determining whether the service request requires the decision comprises providing a list of pending service requests.

13. The method claim 11, wherein providing the recommendation comprises approving or denying the service request including a supporting rationale.

14. The method claim 11, wherein providing the recommendation comprises, upon determining the service request is denied, providing alternate pathways to increase the likelihood the service request is accepted.

15. The method claim 11, wherein providing the recommendation comprises, upon determining the service request is denied, generating a response letter explaining the denial of the service request.

16. The method of claim 11, wherein evaluating the at least one policy against the data comprises training the machine learning model with longitudinal data.

17. The method of claim 1, wherein evaluating the at least one policy against the data comprises comparing the data with the longitudinal data to determine an outcome of the service request.

18. A method for encoding and disambiguating information, the method comprising:
receiving data of a first user;
generating, using a machine learning model, at least one disaggregate criteria for the inclusion, exclusion, or exception of the data; and
storing the disaggregate criteria into a machine comprehensible form for later retrieval.

19. The method of claim 18, wherein the data includes at least one policy associated with at least one treatment.

20. The method of claim 18, wherein the data is a human-readable document.

21. The method of claim 18, wherein storing the disaggregate criteria comprises disaggregating at least one policy into lists of inclusion criteria, exclusion criteria, and exception rules.

22. The method of claim 18, wherein storing the disaggregate criteria comprises assigning concatenating criteria with logical operators.

23. The method of claim 18, wherein storing the disaggregate criteria comprises storing the disaggregate criteria in a checklist.
